(19) United States
(12) Patent Application Publication
(10) Pub. No.: US 2016/0096010 A1
(43) Pub. Date: Apr. 7, 2016

(54) APPLICATOR FOR APPLICATION OF A FLUID SUBSTANCE WITHIN A BODY CAVITY AND METHOD OF USE THEREOF

(71) Applicant: AlgaMed Therapeutics, Rosh Haayin (IL)

(72) Inventor: Ronnie KLEIN, Haifa (IL)

(21) Appl. No.: 14/504,816

(22) Filed: Oct. 2, 2014

(51) Int. Cl. A61M 31/00; A61M 5/315

(52) U.S. Cl. CPC A61M 31/00; A61M 5/315; A61M 5/31501; A61M 2005/3131

(57) ABSTRACT

An applicator device for applying a fluid substance within a body cavity includes an applicator body for insertion into the body cavity. A chamber within the applicator body holds the fluid substance. A plunger applies pressure to the fluid substance in the chamber when pushed into the chamber, and creates suction when withdrawn. Lateral openings are each provided with one-way valves that enable outflow of the fluid substance through the opening from the chamber to an outer surface of the applicator when the pressure is applied by the plunger, and inhibit inflow of a fluid through the opening from outside the applicator into the chamber when the suction is created by the plunger. A stopping structure inhibits pushing motion at a stop position, enables motion past the stop position when an additional pushing force is applied, and enables withdrawal through the stop position without interference.

### FIELD OF THE INVENTION

The present invention relates to applicators. More particularly, the present invention relates to an applicator for application of a fluid substance within a body cavity and a method of using the applicator.

### BACKGROUND OF THE INVENTION

A treatment of a medical condition within a body cavity may include application of a substance to the inside of the cavity. For example, a body cavity may include a vagina, rectum, nostril, ear canal, or another opening or cavity. The applied substance may be in the form of a liquid, cream, or gel.

The substance may be applied to an internal lining of the body cavity. In order to effectively apply the substance to the lining, the applicator may have outer dimensions that are similar to the interior dimensions of the cavity. Thus, the outer surface of the applicator may be in contact with the interior surface of the cavity.

### SUMMARY OF THE INVENTION

There is thus provided, in accordance with an embodiment of the present invention, an applicator device for applying a fluid substance within a body cavity, the device including: an applicator body that is configured for insertion into the body cavity; at least one chamber within the applicator body for holding the fluid substance; a plunger that is configured to apply a pressure to the fluid substance in the chamber when pushed into the chamber with a pushing motion, and to create a suction within the chamber when withdrawn from the chamber with a withdrawal motion; a plurality of lateral openings, each opening provided with a one-way valve that is configured to enable outflow of the fluid substance through the opening from the chamber to an outer surface of the applicator body when the pressure is applied by the plunger, and to inhibit inflow of a fluid through the opening from outside the applicator into the chamber when the suction is created by the plunger; and a stopping structure that is configured to inhibit the pushing motion at a stop position, to enable the pushing motion past the stop position when an additional pushing force is applied, and to enable the withdrawal motion through the stop position without interference.

Furthermore, in accordance with some embodiments of the present invention, the applicator body includes a mouth that is connectable to a supply of the fluid substance such that when the suction is created within the chamber, the fluid substance is drawn through the mouth from the supply and into the chamber.

Furthermore, in accordance with some embodiments of the present invention, the mouth is provided with a valve that enables intake of the fluid substance from the supply into the chamber, and that inhibits outflow of the fluid substance from the chamber through the mouth.

Furthermore, in accordance with some embodiments of the present invention, the mouth is provided with an attachment mechanism to attach the supply to the mouth.

Furthermore, in accordance with some embodiments of the present invention, the attachment mechanism includes threading.

Furthermore, in accordance with some embodiments of the present invention, the applicator body is substantially cylindrical, and the plunger is configured to move substantially along a longitudinal axis of the substantially cylindrical applicator body.

Furthermore, in accordance with some embodiments of the present invention, an end of the applicator body includes a curved surface.

Furthermore, in accordance with some embodiments of the present invention, the one-way valve includes a tapered projection with flexible walls and with an opening at a distal end of the projection.

Furthermore, in accordance with some embodiments of the present invention, the flexible walls include an elastomer.

Furthermore, in accordance with some embodiments of the present invention, the one-way valve is located within an indentation on the outer surface of the applicator.

Furthermore, in accordance with some embodiments of the present invention, the stopping structure is configured to engage a head of the plunger.

Furthermore, in accordance with some embodiments of the present invention, the stop position is placed such that when the plunger is pushed to the stop position, some of the fluid substance flows out of the openings to lubricate the outer surface of the applicator.

Furthermore, in accordance with some embodiments of the present invention, the at least one chamber includes a plurality of separated chambers, each of the separated chambers being provided with a separately operable plunger and stopping mechanism.

Furthermore, in accordance with some embodiments of the present invention, the plurality of separated chambers are separated by one or more longitudinal partitions.

Furthermore, in accordance with some embodiments of the present invention, each of the plurality of separated chambers is provided with a separate mouth to enable filling that separated chamber.

There is further provided, in accordance with some embodiments of the present invention, a method for applying a fluid substance to within a body cavity, the method including: pushing a plunger of an applicator to a stop position to open a one-way valve of each opening of a plurality of lateral openings on the applicator, and to expel a predetermined quantity of the fluid substance from a chamber within the applicator via the plurality of lateral openings to an outer surface of the applicator, so as to lubricate the outer surface; inserting the lubricated surface of the applicator into the body cavity; and pushing the plunger with an additional force to move the plunger past the stop position so as to dispense the fluid substance within the body cavity.

Furthermore, in accordance with some embodiments of the present invention, the method includes pushing an additional plunger of the applicator to a stop position to expel a fluid substance from an additional chamber of the applicator via an additional plurality of lateral openings.

Furthermore, in accordance with some embodiments of the present invention, the fluid substance that is expelled by pushing the plunger of an applicator is different from the fluid substance that is expelled from the additional chamber by pushing the additional plunger.

Furthermore, in accordance with some embodiments of the present invention, the method includes filling the chamber with the fluid substance by connecting a mouth of the applicator to a supply of the fluid substance, and pulling the plunger outward so as to open a one-way valve of the mouth and draw the fluid substance from the container into the applicator via the mouth.

Furthermore, in accordance with some embodiments of the present invention, the method includes filling an additional chamber of the applicator with an additional fluid substance.

### BRIEF DESCRIPTION OF THE DRAWINGS

In order for the present invention, to be better understood and for its practical applications to be appreciated, the following Figures are provided and referenced hereafter. It should be noted that the Figures are given as examples only and in no way limit the scope of the invention. Like components are denoted by like reference numerals.

FIG. 1 schematically illustrates an applicator for applying a substance within a body cavity, in accordance with an embodiment of the present invention.

FIG. 2A is a schematic cross section of the applicator shown in FIG. 1 during filling of the applicator.

FIG. 2B is a schematic cross section of the applicator shown in FIG. 1 at the end of expelling a fluid substance for lubrication.

FIG. 2C is a schematic cross section of the applicator shown in FIG. 1 while dispensing a fluid substance.

FIG. 3A schematically illustrates filling an applicator with a fluid substance, in accordance with an embodiment of the present invention.

FIG. 3B schematically illustrates lubricating the outer surface of a filled applicator, in accordance with an embodiment of the present invention.

FIG. 3C schematically illustrates insertion into a body cavity of an applicator that is lubricated as shown in FIG. 3B.

FIG. 3D schematically illustrates dispensing a fluid substance by an applicator that is inserted into a body cavity as shown in FIG. 3C.

FIG. 4 is a flowchart depicting a method for using an applicator in accordance with an embodiment of the present invention to apply a fluid substance within a body cavity.

FIG. 5 schematically illustrates a divided applicator for separate application of fluid substances, in accordance with an embodiment of the present invention.

### DETAILED DESCRIPTION OF THE INVENTION

In the following detailed description, numerous specific details are set forth in order to provide a thorough understanding of the invention. However, it will be understood by those of ordinary skill in the art that the invention may be practiced without these specific details. In other instances, well-known methods, procedures, components, modules, units and/or circuits have not been described in detail so as not to obscure the invention.

Although embodiments of the invention are not limited in this regard, discussions utilizing terms such as, for example, “processing,” “computing,” “calculating,” “determining,” “establishing”, “analyzing”, “checking”, or the like, may refer to operation(s) and/or process(es) of a computer, a computing platform, a computing system, or other electronic computing device, that manipulates and/or transforms data represented as physical (e.g., electronic) quantities within the computer's registers and/or memories into other data similarly represented as physical quantities within the computer's registers and/or memories or other information non-transitory storage medium (e.g., a memory) that may store instructions to perform operations and/or processes. Although embodiments of the invention are not limited in this regard, the terms “plurality” and “a plurality” as used herein may include, for example, “multiple” or “two or more”. The terms “plurality” or “a plurality” may be used throughout the specification to describe two or more components, devices, elements, units, parameters, or the like. Unless explicitly stated, the method embodiments described herein are not constrained to a particular order or sequence. Additionally, some of the described method embodiments or elements thereof can occur or be performed simultaneously, at the same point in time, or concurrently. Unless otherwise indicated, us of the conjunction “or” as used herein is to be understood as inclusive (any or all of the stated options).

In accordance with an embodiment of the present invention, an applicator is configured for application of a fluid substance, such as a liquid, cream, gel, power, or similar fluid or semi-fluid substance, to the inside of a body cavity. The body cavity may include a vagina, or another body cavity such as a rectum, nostril, ear canal, or other natural or surgically formed body cavity.

The applicator includes an applicator body that is designed for insertion into the body cavity. The applicator body has a generally cylindrical shape with a circular or oval cross section. The outer dimensions of the applicator body are similar to the inner dimensions of the body cavity into which the applicator body is designed for insertion. For example, the outer dimensions of the applicator body may be substantially equal to the inner dimensions of the body cavity. In this case, when the applicator body is inserted into the body cavity, the outer surface of the applicator body is in contact with the inner lining of the body cavity.

The applicator body encloses a chamber that may be filled with the substance. A plunger is provided. The filling mouth is provided with a one-way valve that enables the substance to enter the chamber via the filling mouth. The one-way valve prevents the substance from exiting the chamber via the filling mouth. A filling mouth of the applicator body may be inserted into a container of the substance. Pulling the plunger out of the chamber when the filling mouth is inserted in the container may suck the substance from the container and into the chamber.

A plurality of one-way openings connects the chamber to the outer surface of the applicator body. The one-way openings are located on a lateral wall of the applicator body. As used herein, an opening is considered lateral if it is open in a direction that is substantially perpendicular to a direction of motion of the plunger. For example, the plunger may be configured to move along a longitudinal axis of a cylindrical applicator body. In this case, an opening is considered to be a lateral opening if it is located on a wall of the cylinder, and not on a cap or end of the cylinder.

For example, each one-way opening may be placed at the end of a cone-like outward projection of elastic material. Outward pressure may tend to extend the cone outward and open, or hold open, the opening at the end of the cone. On the other hand, inward pressure may tend to collapse the projections and close the opening. Pushing the plunger into the chamber may apply pressure to the substance in the chamber. Applying pressure to the substance in the chamber may expel some of the substance out of the chamber through the openings to the outer surface of the applicator body.

The plunger is configured to have at least one intermediate stop position. For example, a stop ring, catch, or other stopping mechanism or structure may be configured to resist motion of the plunger when an end of the plunger, or another part of the plunger, reaches the stopping structure. The stop position is located such pushing the plunger to the stop position may expel a part of the substance to the outer surface of the applicator body. The stopping mechanism may operate only when the plunger is pushed inward to expel the substance from the chamber. In this case, when the plunger is pulled outward and withdrawn from the chamber in order to fill the chamber with the substance, the stopping mechanism may allow the plunger to pass without interference. For example, the stopping mechanism may have an asymmetric structure that only stops the plunger during motion in a single direction.

When a part of the substance is expelled to the outer surface of the applicator body, the substance may lubricate the outer surface. Lubrication of the outer surface may reduce friction between the outer surface and the inner lining of the body cavity. Thus lubrication of the outer surface may facilitate insertion of the applicator body into the body cavity. Facilitating insertion of the applicator body into the body cavity may reduce, minimize, or eliminate any discomfort that could otherwise have been associated with the insertion. In some cases, the presence of the substance on the outer surface of the applicator body may facilitate relaxation of muscles that are present within the walls of the body cavity. Relaxation of the muscles may enable natural flexibility and resilience of the walls of the body cavity to further facilitate insertion of the applicator body into the body cavity.

The applicator body may be fully inserted into the body cavity. The applicator body may be considered to be fully inserted when at least some of the openings on the outer surface of the applicator body are situated opposite a target region on the inner wall of the body cavity. For example, the target region may represent a region of the body cavity where the substance is to be placed or delivered for a therapeutic effect. For example, the therapeutic effect may include contributing to curing a disorder within the body cavity or in adjacent tissue, preventing or inhibiting a disorder (e.g., by cleansing, disinfecting, or moisturizing the body cavity or adjacent tissue, or otherwise providing a prophylactic effect), deodorizing or providing another cosmetic or esthetic benefit, a contraceptive effect, or other therapeutic effect.

When the applicator body is fully inserted into the body cavity, some or all of the substance that remains in the chamber may be ejected. An additional force (greater than the force that is applied during lubrication) may be applied to the plunger to push the plunger past one or more stop positions. Pushing the plunger past the stop positions causes further expelling of the substance from the chamber and via the openings to the outer surface off the applicator body. The further expelled substance on the outer surface may be dispensed to the inner wall of the body cavity.

The applicator may be disposable, designed to be discarded after a single use. For example, the applicator may be manufactured under clean or sterile conditions and enclosed in a sealed package until removed for use. Alternatively or in addition, an applicator may be designed for at least limited reuse. For example, components of the applicator may be designed for at least partial disassembly for cleaning or disinfection, and for reassembly prior to use. In this case, the components of applicator may be design to withstand exposure to the conditions of cleaning or disinfection (e.g., high temperatures, chemical agents, ultraviolet radiation, or other conditions).

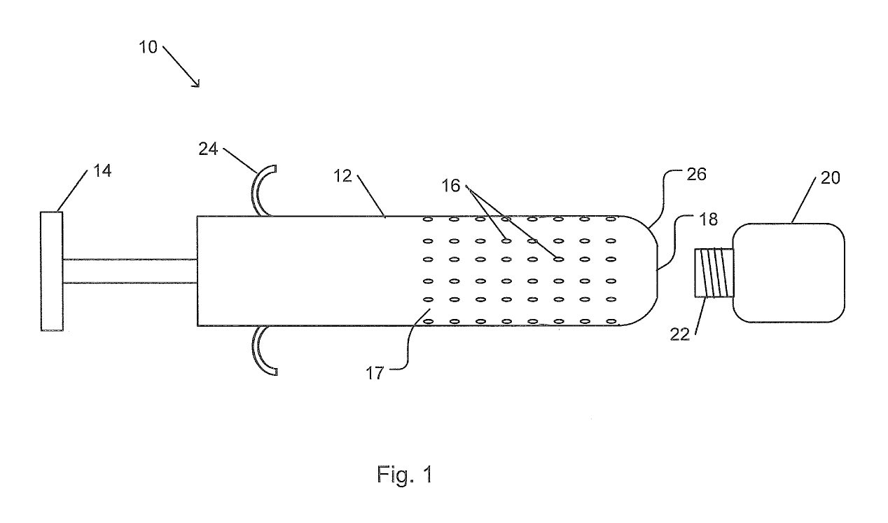

FIG. 1 schematically illustrates an applicator for applying a substance within a body cavity, in accordance with an embodiment of the present invention.

Applicator 10 includes applicator body 12. Plunger 14 is configured to be pushed into, or pulled out of, applicator body 12. A size and shape of applicator body 12 may be designed to enable or facilitate insertion of applicator body 12 within a body cavity, such as a vagina, rectum, ear canal, nostril, or other body cavity that opens to the outside of a human or animal body. For example, an applicator body 12 that is designed for insertion into a vagina or rectum may have an elongated cylindrical shape, with a substantially circular or oval cross section.

Applicator body 12 may include a curved surface 26 at an end of applicator body 12 that is designed to be inserted into a body cavity. Curved surface 26 may be shaped to facilitate insertion in the body cavity, or to prevent injury or discomfort during insertion into the body cavity.

A substance that is held interior to applicator body 12 may be expelled to applicator outer surface 17 via lateral openings 16. (The terms “expelling” and “dispensing” as used herein should be understood as interchangeable.) For example, lateral openings 16 may be distributed in a pattern on applicator outer surface 17.

Lateral openings 16 are one-way openings that enable substance to be expelled via lateral openings 16 but prevent intake of the substance or another fluid material into the interior of applicator body 12 via lateral openings 16. For example, a portion of applicator outer surface 17 that includes lateral openings 16 may be made of an elastic material.

Applicator mouth 18 enables introduction of a fluid substance into applicator body 12. Applicator mouth 18 may be equipped with a one-way valve that enables introduction of the fluid substance, but prevents expelling of the material via applicator mouth 18. Applicator mouth 18 may be configured to connect to an opening of a supply of the fluid substance, such as container 20. Applicator mouth may be provided with structure that cooperates with corresponding structure on container 20 to attach or hold container 20 to applicator mouth 18 while filling applicator body 12 with the fluid substance. For example, applicator mouth 18 may be provided with an interiorly threaded inner surface that cooperates with exterior threading 22 on a neck of container 20. In some cases, applicator mouth 18 may be provided with a neck with external threading inner surface that mates with interior threading in an opening of container 20. Alternatively or in addition, the cooperating structure may include structure for a bayonet mount, a clamp, a pin, or other cooperating structure. Alternatively or in addition, applicator mouth 18 or container 20 may be provided with a sealing ring or other sealing structure to enable a snug fit between container 20 and applicator mouth 18. Instead of container 20, a supply of the fluid substance may include a hose, tube, pipe, tap, nozzle, or other arrangement that is capable of delivering the fluid substance to applicator mouth 18.

Plunger 14 may be pulled with withdrawal motion 40 until withdrawal motion 40 is stopped by contact of plunger head 15 with rear wall 12a of applicator body 12. Alternatively or in addition, withdrawal motion 40 of plunger 14 may be stopped by other structure of plunger 14 engaging other structure of applicator body 12.

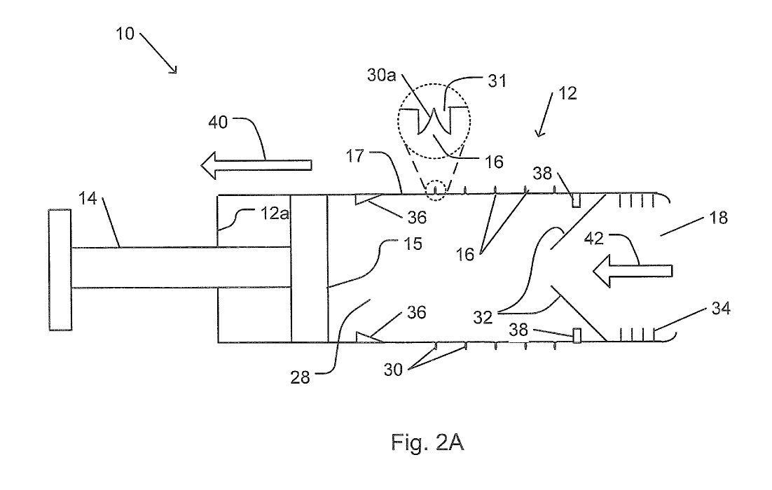

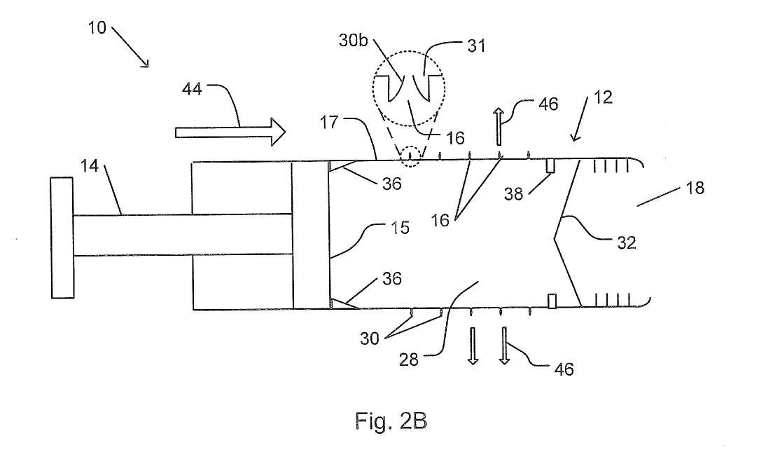

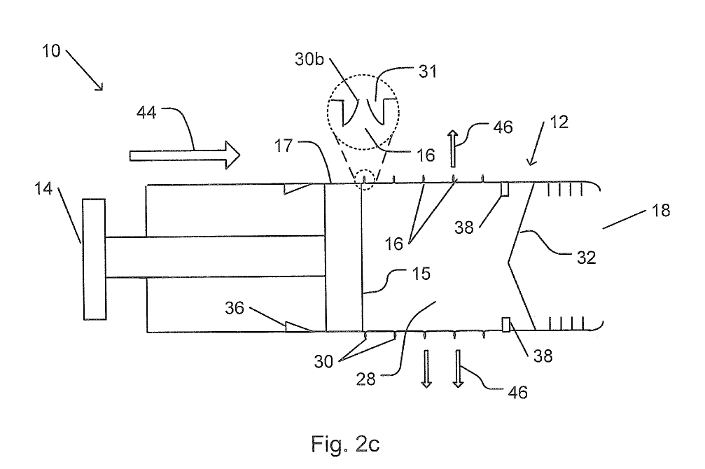

Thus, when container 20 is connected to applicator mouth 18, pulling plunger 14 outward may draw the fluid substance from container 20 into applicator body 12 (e.g., into substance chamber 28 in applicator body 12, as shown in FIG. 2A). After filling applicator body 12, container 20 may be detached or removed from applicator mouth 18. On the other hand, pushing plunger 14 inward may force the fluid substance out of applicator body 12 through lateral openings 16 to applicator outer surface 17. Grasping structure 24 may be provided to facilitate application of a pushing or pulling force to plunger 14. Grasping structure 24 may also facilitate insertion of applicator body 12 into a body cavity, or removal of applicator body from a body cavity. For example, grasping structure 24 may include a handle, groove, ring, projection, friction surface, or other structure to enable application of a counterforce when pushing or pulling on plunger 14.

Filling of applicator body 12 with a fluid substance from container 20 may be further assisted by application of slight pressure to container 20. For example, container 20 may include a squeezable tube, bottle, bag, balloon, or other squeezable element. Container 20 may include a pump, piston, plunger, or other mechanism to apply pressure to a fluid substance within container 20.

Each lateral opening 16 is provided with a one-way valve 30 (FIGS. 2A and 2B). Each one-way valve 30 is configured to enable outflow of the fluid substance from applicator body 12 through a lateral opening 16 to applicator outer surface 17. Each one-way valve 30 is further configured to prevent or inhibit inflow of a fluid from outside applicator body 12 (e.g., within a body cavity) through a lateral opening 16 into applicator body 12. For example, applicator outer surface 17 may include an elastomer layer or surface. At the point of each lateral opening 16, the elastomer surface may be shaped in the form of a tapered (e.g., cone-like or volcano-shaped) projection with an opening at its distal end. The walls of the projection may collapse under inward pressure, thus closing the opening Outward pressure, on the other hand, may open the opening, enabling an outward flow of the fluid substance.

FIG. 2A is a schematic cross section of the applicator shown in FIG. 1 during filling of the applicator.

During filling of applicator 10, a supply of a fluid substance may be connected or attached to applicator mouth 18. For example, a neck or nozzle of the supply of the fluid substance may have exterior threading (e.g., exterior threading 22 as shown in FIG. 1) that mates with interior threading 34 in applicator mouth 18 to attach the supply to applicator mouth 18. Alternatively or in addition, a force that is applied (e.g., by a person holding the supply of fluid substance to applicator mouth 18, or a suction that is form by motion of plunger 14) may hold a neck of the supply to applicator mouth 18. Other connectors or attachment mechanisms or arrangements may be used to attach the supply to applicator mouth 18.

Plunger 14 may be pulled outward as indicated by withdrawal motion 40. In pulling plunger 14 with withdrawal motion 40, plunger head 15 may be pulled past lubrication stop structure 36 in substance chamber 28 without impedance. For example, lubrication stop structure 36 may be configured such that motion of plunger head 15 with withdrawal motion 40 causes lubrication stop structure 36 to retract to within a wall of substance chamber 28. A slope on a surface of a catch or tab of lubrication stop structure 36 that engages plunger head 15 (or other cooperating structure of plunger 14) may be sufficiently shallow so as not to catch. Lubrication stop structure 36 may be further configured such that after plunger head 15 has passed lubrication stop structure 36, lubrication stop structure 36 is re-extended (e.g., by a spring or other resilient structure).

Pulling plunger 14 outward withdraws plunger head 15 away from applicator mouth 18 within substance chamber 28. The outward motion of plunger head 15 may create suction within substance chamber 28.

The suction may open mouth valve 32. For example, mouth valve 32 may include a passive (e.g., hinged or flexible) one-way valve that may open or close in accordance with whether negative pressure (suction) or positive pressure is applied to substance chamber 28. As another example, mouth valve 32 may include an elastic or resilient one-way valve, or may be provided with an elastic or resilient spring or other member that holds mouth valve 32 in the absence of suction within substance chamber 32. Mouth valve 32 is configured to enable intake of the fluid substance from the supply of the fluid substance into substance chamber 28. Mouth valve 32 is also configured to prevent or inhibit outflow of the fluid substance from substance chamber 28 via applicator mouth 18. Thus, the suction may draw the fluid substance from the substance supply into substance chamber 28 via applicator mouth 18 and open mouth valve 32, as indicated by filling flow 42.

Each lateral opening 16 is provided with a one-way valve 30. When suction is applied to substance chamber 28, one-way valves 30 are closed, as indicated by closed valve state 30a. When one-way valves 30 are in closed valve state 30a, flow from outside of substance chamber 28 into substance chamber 28 is prevented or inhibited. Thus, ambient fluids, such as atmospheric air, cannot be drawn into substance chamber 28 via lateral openings 16 when filling substance chamber 28 via applicator mouth 18. Thus, suction may be maintained during the filling process.

For example, each one-way valve 30 may include a tapered projection with flexible or elastic walls, and an opening at the distal end of the projection. Thus, an inward force, as caused by suction within substance chamber 28, may cause the walls of the tapered projection to collapse inward, thus sealing the opening. In this case, one-way valve 30 is in closed state 30a.

As a further advantage of one-way valves 30, plunger 14 may be accidentally or unintentionally pulled with withdrawal motion 40 when applicator body 12 is inserted into a body cavity. In this case, closing of one-way valves 30 may prevent ambient fluids from the body cavity from being drawn into substance chamber 28. Accidental introduction of ambient fluids into substance chamber 28 via applicator mouth 18 may be prevented by placement of a cap, plug, or cover on applicator mouth 18 after filling substance chamber 28.

Each one-way valve 30 may be located within an indentation 31 in applicator outer surface 17. For example, location within indentation 31 may ensure that the distal ends of one-way valves 30 do not extend outward beyond applicator outer surface 17. Ensuring that ends of one-way valves 30 do not extend outward beyond applicator outer surface 17 may prevent contact or friction between one-way valves 30 and an inner wall of a body cavity. Prevention of contact or friction may prevent opening of one-way valves 30 by the contact or friction, or irritation of the body cavity wall.

FIG. 2B is a schematic cross section of the applicator shown in FIG. 1 at the end of expelling a fluid substance for lubrication.

As shown in FIG. 2B, plunger 14 has been moved with pushing motion 44 to push plunger head 15 up to lubrication stop structure 36. During pushing motion 44, plunger head 15 applies pressure to the fluid substance that is within substance chamber 28. The pressure forces mouth valve 32 to close, thus preventing or inhibiting expelling of the fluid substance via applicator mouth 18. The pressure forces one-way valves 30 to open to open valve state 30b. When in one-way valves 30 are in open valve state 30b, the fluid substance may flow outward via lateral openings 16 with substance expulsion flow 46. The fluid substance that is dispensed or expelled via lateral openings 16 may coat and lubricate applicator outer surface 17.

For example, as described above, one-way valve 30 may include a tapered projection with flexible or elastic walls and with opening at the distal end of the projection. Thus, an outward force, as caused by pressure within substance chamber 28, may cause the walls of the tapered projection to spread outward, thus holding the opening in an open position. In this case, one-way valve 30 is in open state 30b.

Lubrication stop structure 36 is configured to stop or impede pushing motion 44 of plunger 14 at a stop position. As shown, lubrication stop structure 36 engages plunger head 15 to impede pushing motion 44. For example, an edge of a catch of lubrication stop structure 36 may be substantially flat and parallel to plunger head 15 such that a force (weaker than a threshold force) in the direction of pushing motion 44 does not cause the catch to retract. Alternatively or in addition, one or more stop structures may be configured to engage structure on other parts of plunger 15 (e.g., one or more projections, bosses, notches, depressions, rims, grooves, lips, tabs, or other structure) to impede pushing motion 44.

A stop position of plunger head 15 within substance chamber 28 as determined by lubrication stop structure 36 may be selected to impede pushing motion 44 after a predetermined quantity of the fluid substance has been expelled (e.g., after movement from an initial position after filling). The expelled quantity may be such as is expected to sufficiently lubricate applicator outer surface 17. More than one stop position may be provided. For example, various stop positions may be provided to accommodate different degrees of filling of substance chamber 28, or different types of fluid substance. As another example, moving plunger head 15 from one stop position to an adjacent or next stop position may expel the predetermined quantity of the fluid substance.

Lubrication stop structure 36 is configured such that, when sufficient force is applied to plunger 14, plunger head 15 may continue to move with pushing motion 44 past the stop position that is determined by lubrication stop structure 36. For example, an edge or projection of a catch of lubrication stop structure 36 that is configured to engage corresponding structure of plunger 14 (e.g., is substantially parallel to an engaging end of plunger head 15) may include a bevel or curve, or may be sloped. Thus, when a relatively small force (e.g., not exceeding a threshold force) is applied to plunger 14, motion of plunger 14 is impeded by lubrication stop structure 36. When a larger pushing force (e.g., exceeding a threshold force) is applied to plunger 14, plunger 14 may continue to move with pushing motion 44.

FIG. 2C is a schematic cross section of the applicator shown in FIG. 1 while dispensing a fluid substance.

As shown in FIG. 2C, plunger 14 has been pushed past a stop position. For example, plunger head 15 may be moving with pushing motion 44 past a stop position as determined by lubrication stop structure 36. Motion of plunger head 15 may continue until ultimately stopped by contact with mouth valve 32. Alternatively or in addition, motion of plunger head 15 may be stopped by contact with end stop 38, or with another structure within substance chamber 28 (e.g., a wall, ring, or lip near applicator mouth 18).

During motion of plunger head 15 past the stop position, all or some of the fluid substance remaining in substance chamber 28 may be dispensed with substance expulsion flow 46 via lateral openings 16 and one-way valves 30 (in open valve state 30b). For example, plunger head 15 may be moved past the stop position after the lubricated applicator body 12 has been fully inserted into a body cavity (e.g., vagina, rectum, or other body cavity). Further dispensing of the fluid substance may be intended to have a therapeutic effect on the body cavity, or on tissue that is adjacent to the body cavity.

Applicator 10 may be utilized to apply a substance within a body cavity.

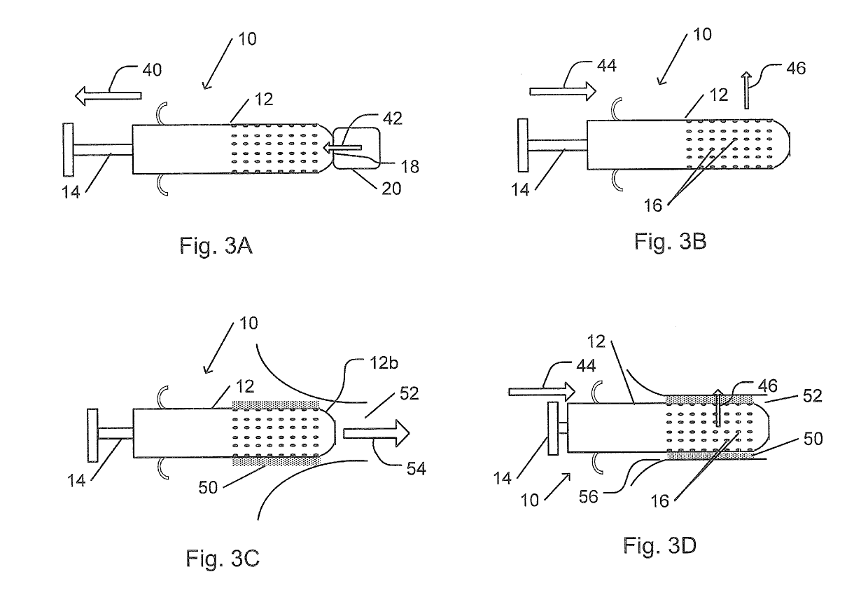

FIG. 3A schematically illustrates filling an applicator with a fluid substance, in accordance with an embodiment of the present invention. For example, the applicator may be filled prior to using the applicator to apply the fluid substance within a body cavity.

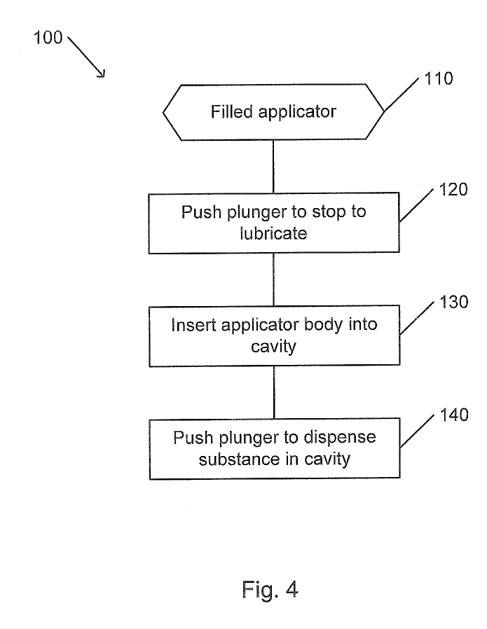

FIG. 4 is a flowchart depicting a method for using an applicator in accordance with an embodiment of the present invention to apply a fluid substance within a body cavity.

It should be understood with respect to any flowchart referenced herein that the division of the illustrated method into discrete operations represented by blocks of the flowchart has been selected for convenience and clarity only. Alternative division of the illustrated method into discrete operations is possible with equivalent results. Such alternative division of the illustrated method into discrete operations should be understood as representing other embodiments of the illustrated method.

Similarly, it should be understood that, unless indicated otherwise, the illustrated order of execution of the operations represented by blocks of any flowchart referenced herein has been selected for convenience and clarity only. Operations of the illustrated method may be executed in an alternative order, or concurrently, with equivalent results. Such reordering of operations of the illustrated method should be understood as representing other embodiments of the illustrated method.

Substance application method 100 may be performed to apply a fluid substance within a body cavity using a filled applicator 10 (block 110). For example, an applicator 10 may have been filled as illustrated in FIG. 3A.

Container 20, which contains a fluid substance with which applicator body 12 (e.g., a substance chamber 28, as shown in FIG. 2A) is to be filled, is connected to applicator mouth 18 of applicator body 18. For example, container 20 may be connected to applicator mouth by threading, by an applied force, or otherwise. Plunger 18 is pulled with withdrawal motion 40. The resulting suction draws some or all of the fluid substance in accordance with filling flow 42 from container 20 into applicator body 12 via applicator mouth 18. During filling, a mouth valve 32 (FIG. 2A) may be opened by the suction, and one-way valves 30 (FIG. 2A) may be closed by the suction.

After filling applicator body 12, container 20 may be disconnected from applicator mouth 18. Mouth valve 32 may prevent the fluid material from leaking out of applicator body 12.

Plunger 14 of applicator 10 may be pushed to a stop position in order to expel a quantity of the fluid substance that is sufficient to lubricate the outer surface of applicator body 12 (block 120).

FIG. 3B schematically illustrates lubricating the outer surface of a filled applicator, in accordance with an embodiment of the present invention.

Pushing on plunger 14 with pushing motion 44 until a stop position (e.g., as determined by lubrication stop structure 36 as shown in FIG. 2B) applies pressure to the fluid substance within applicator body 12. The applied pressure may cause a quantity of the fluid substance to be expelled from within applicator body 12 with substance expulsion flow 46 via lateral openings 16. The quantity may be sufficient to lubricate the outer surface of applicator body 12. When expelling the substance, mouth valve 32 (FIG. 2A) may be closed by the applied pressure and one-way valves 30 (FIG. 2A) may be opened by the applied pressure to enable flow through lateral openings 16.

The lubricated applicator body 12 may be inserted into a body cavity (block 130).

FIG. 3C schematically illustrates insertion into a body cavity of an applicator that is lubricated as shown in FIG. 3B.

Applicator body 12 is coated with a layer of the fluid substance in the form of lubrication layer 50. Front end 12b of applicator body 12 may be inserted into body cavity 52 with insertion motion 54. During insertion, plunger 14 may remain stationary with respect to applicator body 12. Insertion motion 54 may be facilitated by the presence of lubrication layer 50 on applicator body 12. In some cases, a need for further lubrication may be felt during insertion of applicator body 12 into body cavity 52. In this case, plunger 14 may be further pushed into applicator body 12 in order to add an additional quantity of the fluid substance to lubrication layer 50.

When applicator body 12 is inserted into body cavity 52, plunger 14 may be further pushed to dispense the fluid substance within the body cavity (block 140).

FIG. 3D schematically illustrates dispensing a fluid substance by an applicator that is inserted into a body cavity as shown in FIG. 3C.

Pushing on plunger 14 with pushing motion 44 past a stop position further applies pressure to the fluid substance within applicator body 12. The applied pressure may cause the fluid substance to be dispensed to cavity wall 56 of body cavity 52 from within applicator body 12. The fluid substance is dispensed with substance expulsion flow 46 via lateral openings 16. When dispensing the substance, mouth valve 32 (FIG. 2A) may be closed by the applied pressure and one-way valves 30 (FIG. 2A) may be opened by the applied pressure to enable expulsion flow 46 through lateral openings 16.

The dispensed fluid substance may have a therapeutic effect on cavity wall 56 or on adjacent tissue. Depending on where within body cavity 52 the fluid substance is to be dispensed, the fluid substance may be dispensed when applicator body 12 is fully or partially inserted into body cavity 52.

In accordance with an embodiment of the present invention, an applicator may include one or more additional substance chambers. For example, the applicator body may be divided lengthwise by one or more longitudinal partitions into a plurality of separated substance chambers. Additional substance chambers may enable separate filling with additional fluid substances, which may be identical to one another or different from one another. Additional substance chambers may enable separate expelling or dispensing of additional fluid substances out of different sides of the applicator body. Each of the separated substance chamber is provided with a separately operable plunger. The additional fluid substances that are separately expelled or dispensed may have a single composition (e.g., to enable dispensing different quantities of a single fluid substance to different sides of a body cavity), or different compositions (e.g., to enable dispensing of separate substances within a body cavity).

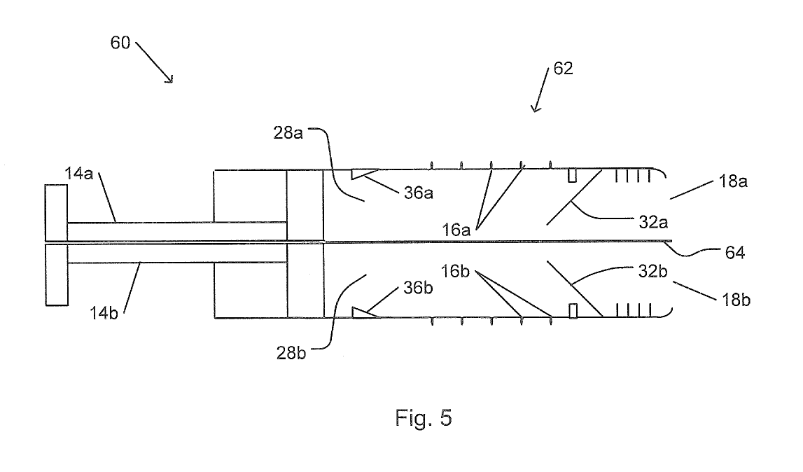

FIG. 5 schematically illustrates a divided applicator for separate application of fluid substances, in accordance with an embodiment of the present invention.

Applicator body 62 of divided applicator 60 divided by lengthwise partition 64 into separate substance chambers 28a and 28b. For example, lengthwise partition 64 may include a single flat partition, as shown. In this case each of separate substance chambers 28a and 28b may have a substantially semicircular cross section. Separate substance chambers 28a and 28b may be substantially equal in volume (as shown), or may have different volumes.

In other cases, a lengthwise partition may include three or more partitions that intersect along an axis of applicator body 62 to form three or more separate substance chambers. The separate substance chambers may have approximately equal volumes or different volumes. Each of the separate substance chambers may have a cross section that is substantially in the form of a sector of a circle,

Each of separate substance chambers 28a and 28b is provided with a chamber mouth 18a and 18b and chamber mouth valve 32a and 32b, respectively. For example, a container having an appropriately shaped neck (e.g., semicircular for divided applicator 60 as shown, or another appropriate circular sector or other shape) may be inserted into one of chamber mouths 18a and 18b to transfer the substance to one of separate substance chambers 28a and 28b, respectively.

Each of separate substance chambers 28a and 28b is provided with a separately operable plunger 14a or 14b, respectively, and a separate lubrication stop structure 36a or 36b, respectively. Separately operable plungers 14a and 14b may be pulled separately or together in order to fill separate substance chambers 28a and 28b either separately or together.

Each of separate substance chambers 28a and 28b is provided with separate lateral openings 16a and 16b, respectively, through which a fluid substance in each of separate substance chambers 28a and 28b may be expelled or dispensed. Separately operable plungers 14a and 14b may be pushed separately or together in order to expel or dispense a fluid substance from one or both of fill separate substance chambers 28a and 28b.

During expelling or dispensing of a fluid substance from one of separate substance chambers 28a and 28b within a body cavity, or when expelling or dispensing different fluid substances from separate substance chambers 28a and 28b within a body cavity, divided applicator may be rotated about its (longitudinal) axis in order to facilitate even distribution of the fluid substance within the body cavity.

Different embodiments are disclosed herein. Features of certain embodiments may be combined with features of other embodiments; thus certain embodiments may be combinations of features of multiple embodiments. The foregoing description of the embodiments of the invention has been presented for the purposes of illustration and description. It is not intended to be exhaustive or to limit the invention to the precise form disclosed. It should be appreciated by persons skilled in the art that many modifications, variations, substitutions, changes, and equivalents are possible in light of the above teaching. It is, therefore, to be understood that the appended claims are intended to cover all such modifications and changes as fall within the true spirit of the invention.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. An applicator device for applying a fluid substance within a body cavity, the device comprising:
an applicator body that is configured for insertion into the body cavity;
at least one chamber within the applicator body for holding the fluid substance;
a plunger that is configured to apply a pressure to the fluid substance in the chamber when pushed into the chamber with a pushing motion, and to create a suction within the chamber when withdrawn from the chamber with a withdrawal motion;
a plurality of lateral openings, each opening provided with a one-way valve that is configured to enable outflow of the fluid substance through the opening from the chamber to an outer surface of the applicator body when the pressure is applied by the plunger, and to inhibit inflow of a fluid through the opening from outside the applicator into the chamber when the suction is created by the plunger; and
a stopping structure that is configured to inhibit the pushing motion at a stop position, to enable the pushing motion past the stop position when an additional pushing force is applied, and to enable the withdrawal motion through the stop position without interference.

2. The device of claim 1, wherein the applicator body includes a mouth that is connectable to a supply of the fluid substance such that when the suction is created within the chamber, the fluid substance is drawn through the mouth from the supply and into the chamber.

3. The device of claim 2, wherein the mouth is provided with a valve that enables intake of the fluid substance from the supply into the chamber, and that inhibits outflow of the fluid substance from the chamber through the mouth.

4. The device of claim 2, wherein the mouth is provided with an attachment mechanism to attach the supply to the mouth.

5. The device of claim 4, wherein the attachment mechanism comprises threading.

6. The device of claim 1, wherein the applicator body is substantially cylindrical and the plunger is configured to move substantially along a longitudinal axis of the substantially cylindrical applicator body.

7. The device of claim 1, wherein an end of the applicator body includes a curved surface.

8. The device of claim 1, wherein the one-way valve comprises a tapered projection with flexible walls and with an opening at a distal end of the projection.

9. The device of claim 8, wherein the flexible walls comprise an elastomer.

10. The device of claim 8, wherein the one-way valve is located within an indentation on the outer surface of the applicator.

11. The device of claim 1, wherein the stopping structure is configured to engage a head of the plunger.

12. The device of claim 1, wherein the stop position is placed such that when the plunger is pushed to the stop position, some of the fluid substance flows out of the openings to lubricate the outer surface of the applicator body.

13. The device of claim 1, wherein said at least one chamber comprises a plurality of separated chambers, each of the separated chambers being provided with a separately operable plunger and stopping mechanism.

14. The device of claim 13, wherein said plurality of separated chambers are separated by one or more longitudinal partitions.

15. The device of claim 13, wherein each of said plurality of separated chambers is provided with a separate mouth to enable filling that separated chamber.

16. A method for applying a fluid substance to within a body cavity, the method comprising:
pushing a plunger of an applicator to a stop position to open a one-way valve of each opening of a plurality of lateral openings on the applicator, and to expel a predetermined quantity of the fluid substance from a chamber within the applicator via said plurality of lateral openings to an outer surface of the applicator, so as to lubricate the outer surface;
inserting the lubricated surface of the applicator into the body cavity; and
pushing the plunger with an additional force to move the plunger past the stop position so as to dispense the fluid substance within the body cavity.

17. The method of claim 16, further comprising pushing an additional plunger of the applicator to a stop position to expel a fluid substance from an additional chamber of the applicator via an additional plurality of lateral openings.

18. The method of claim 17, wherein the fluid substance that is expelled by pushing said plunger of an applicator is different from the fluid substance that is expelled from the additional chamber by pushing the additional plunger.

19. The method of claim 16, further comprising filling the chamber with the fluid substance by connecting a mouth of the applicator to a supply of the fluid substance, and pulling the plunger outward so as to open a one-way valve of the mouth and draw the fluid substance from the container into the applicator via the mouth.

20. The method of claim 19, further comprising filling an additional chamber of the applicator with an additional fluid substance.
